(19) United States
(12) Patent Application Publication
(10) Pub. No.: US 2021/0207082 A1
(43) Pub. Date: Jul. 8, 2021

(54) SPHERICAL 3D TUMOR SPHEROID

(71) Applicant: KOREA INSTITUTE OF SCIENCE AND TECHNOLOGY, Seoul (KR)

(72) Inventors: Sang-Heon KIM, Seoul (KR); Seung Ja OH, Seoul (KR); Wooshik CHOI, Seoul (KR); Inyeong BAE, Seoul (KR)

(73) Assignee: KOREA INSTITUTE OF SCIENCE AND TECHNOLOGY, Seoul (KR)

(21) Appl. No.: 17/127,545

(22) Filed: Dec. 18, 2020

(30) Foreign Application Priority Data
Dec. 20, 2019 (KR) 10-2019-0172372
Dec. 16, 2020 (KR) 10-2020-0176595

(51) Int. Cl. C12N 5/00; C12N 5/09; G01N 33/50

(52) U.S. Cl. CPC C12N 5/0062; C12N 5/0693; G01N 33/5044; G01N 2800/52; C12N 2533/52; C12N 2533/54; C12N 2502/30; C12N 2502/1382; C12N 2513/00

(57) ABSTRACT

A spherical 3D tumor spheroid according to an aspect has an appropriate diameter, roundness and specificity so as to be suitably used in vitro, and expresses an ECM structure similar to that of in vivo tumor, and thus may be used in evaluating the efficacy of drug for treating various types of tumors.

### CROSS-REFERENCE TO RELATED APPLICATION

This application is based on and claims priority under 35 U.S.C. § 119 to Korean Patent Application No. 10-2019-0172372, filed on Dec. 20, 2019, in the Korean Intellectual Property Office, the disclosure of which is incorporated by reference herein in its entirety.

### BACKGROUND

### 1. Field

The present disclosure relates to a spherical 3D tumor spheroid and a method for evaluating efficacy of a tumor therapeutic agent using the same.

### 2. Description of Related Art

Currently, most of pre-clinical tests in vitro for development of new anticancer drugs are conducted using cells cultured in typical tissue culture plates of a two dimensional (2D) environment. To obtain results prior to in vivo tests for drug development, presumably more than 70% of cancer researchers still depend on two dimensional (2D) culture systems. However, in vitro cells of 2D culture systems grow on a planar surface as a monolayer, and thus may not be suitable for drug screening, making it difficult to accurately reflect cells existing in an in vivo three dimensional (3D) environment. In this regard, the predictive efficacy of in vivo drug evaluation has proven to be insufficient, resulting in a costly failure in clinical tests due to a huge financial loss.

To overcome limitations of 2D culture systems in pre-clinical stages, attention has recently been focused on 3D culture technique as a tool for improving physiological relevance of in vitro models for evaluating drug efficacy. The in vitro models equipped with 3D platforms, including multicellular spheroids, scaffolds and biochips, have been developed in many researches. The 3D culture platform has led to development of new, more physiological human normal tissues and tumor models. Many researchers have established in vitro models of tumors and living organs associated with drug metabolism of a human body, including liver, heart, viscera and kidney.

### SUMMARY

An object of the present disclosure is to provide a spherical 3D tumor spheroid and a method for evaluating efficacy of a tumor therapeutic agent using the same.

Additional aspects will be set forth in part in the description which follows and, in part, will be apparent from the description, or may be learned by practice of the presented embodiments of the disclosure.

An aspect provides a spherical 3D tumor spheroid comprising a core part including a tumor cell group; and a peripheral part comprising an adipose-derived stromal cell group and an extracellular matrix component and surrounding the core part.

Another aspect provides a tumor cell model including the 3D tumor spheroid.

Another aspect provides a screening composition including the cell model.

Another aspect provides a composition for evaluating a tumor therapeutic agent comprising the 3D tumor spheroid.

The cells of the tumor cell group and the adipose-derived stromal cell group are not particularly limited so long as the cells are derived from individuals having tissues from which tumors may be generated, and examples thereof may include cells derived from humans, mice, rabbits, dogs, pigs or monkeys, specifically from humans.

The cells of the tumor cell group are not particularly limited so long as the cells are obtained from tumor cell lines that can be easily available in the art. For examples the tumor cell group comprising, but not limited to, a breast cancer cell, a lung cancer cell, a fibrosarcoma cell, a stomach cancer cell, an oral cavity cancer cell, a prostate cancer cell, a liver cancer cell, an ovarian cancer cell, a thyroid cancer cell, a uterine cancer cell, a glioblastoma cell, a melanoma cell, a gingiva cancer cell, a tongue cancer cell, a pancreatic cancer cell, a kidney cancer cell, a bone cancer cell, a testicular cancer cell, a mesothelioma cell, a lymphoma cell, a brain tumor cell, a colon cancer cell or a bladder cancer cell.

According to a specific embodiment, the tumor cell group may include MDA-MB-231, MDA-MB-468, BT-549, MCF-10A, MCF-7, A549, HT1080, MKN45 or SK-BR-3 cell.

The extracellular matrix component may be a product expressed by an interaction between the tumor cell group and the adipose-derived stromal cell group. The extracellular matrix component may include components existing in the entire peripheral part, including a surface of the spheroid, and may be configured to surround the entire core part. When an expression level of the extracellular matrix component is low, the extracellular matrix component may not be implemented so as to be similar to an actual tumor tissue known to have an over-expressed extracellular matrix component, and thus making it difficult to use the extracellular matrix component in screening a cancer or tumor therapeutic agent or evaluating efficacy thereof.

The extracellular matrix component may include collagen and fibronectin. The extracellular matrix component may be a product expressed by an interaction between the tumor cell group and the adipose-derived stromal cell group, as described above. The collagen may include collagen type 1, which plays a key role in preventing penetration of a cancer or tumor therapeutic agent. Thus, the collagen can serve as a main checkpoint in evaluating or screening efficacy of the cancer or tumor therapeutic agent using the spheroid.

The spheroid may have a diameter of 200 μm to 900 μm, 250 μm to 850 μm, 300 μm to 800 μm, 350 μm to 750 μm, 400 μm to 700 μm, 450 μm to 650 μm, or 500 μm to 600 μm, and the diameter of the spheroid is preferably 500 μm to 600 μm. When the diameter of the spheroid is within this range, the 3D tumor spheroid is able to similarly mimic an in vivo tumor while maintaining an appropriate size as an in vitro 3D tumor spheroid.

The spheroid may have a roundness of 0.90 or greater. In addition, the spheroid may have a sphericity of 0.90 or greater. Preferably, the roundness of the spheroid is 0.99 or greater and the sphericity thereof is 0.99 or greater. To be used as a model for drug efficacy evaluation or drug screening, it is very important to stably form a spheroid having the same type and shape. Given that uniformity and consistency in forming a spheroid are very important particularly for establishing metabolic activity, the spheroid may have a perfect, uniform, and stable spherical shape.

The spheroid may be formed by co-culturing the tumor cell group and the adipose-derived stromal cell group at a cell density of 7:3 to 3:7, preferably 1:1, which may be based on the effect of the spheroid having increasing viability and decreasing drug permeability as the cell density is closer to 1:1. In such a case, by using a spheroid having the tumor cell group and the adipose-derived stromal cell group co-cultured at an appropriate cell density ratio within the range of 7:3 to 3:7, the efficacy of a cancer or tumor therapeutic agent may be screened or comparatively tested. As an example of the comparative test, when the co-cultured cell density ratio of the tumor cell group and the adipose-derived stromal cell group is 7:3 or 1:1, whether or not the spheroid penetrates the core of the candidate drug can be observed. For example, when the first drug succeeds in penetrating into the core portion only at 7:3, and the second drug succeeds in penetrating into the core portion in both 7:3 and 1:1 cases, it is determined that the first drug has lower efficacy than the second drug in treating a cancer or tumor.

In a specific embodiment, provided is a method for evaluating efficacy of a cancer or tumor therapeutic agent comprising: treating target drugs with the spheroid; and analyzing distributions of the target drugs in a core part of the spheroid or analyzing cell viability in the spheroid.

In treating the drugs, a general method used in the art for evaluating the efficacy of a cancer or tumor therapeutic agent in vitro, or a method of adding a cancer or tumor therapeutic agent to may be used.

For example, the treatment of the drug may be in the form of a culture medium having a spheroid seeded therein for a predetermined period of time and then culturing.

In treating the target drugs, a plurality of drugs may be treated on a plurality of spheroids in one-to-one (1:1) correspondence, or a plurality of drugs may be treated on a single spheroid, but not limited thereto. Any method may be employed as long as there is no problem in performance of the following step of analyzing distributions of the target drugs in a core part of the spheroid or analyzing cell viability in the spheroid.

In analyzing a distribution of the target drug in the core part of each spheroid, any method that is known in the art as a method for tracking a moving path of the target drug or an extent of distribution thereof, may be used without particular limitation, and, for example, the distribution may be analyzed by fluorescence detected from the drug.

In analyzing cell viability in the spheroid, the viability may be analyzed by counting the number of cells living in the spheroid before and after drug treatment, but any method that is known in the art as a method for analyzing cell viability may be employed without any particular limitation.

The cell viability in the spheroid reflects the overall phenomenon in which the size or number of tumor cell groups according to treatment of the target drug. That is, the cell viability may reflect diverse and comprehensive phenomena including apoptosis, necrosis, etc.

Considering selective therapy on tumor cells of the cancer or tumor therapeutic agent, the viability of cells in the spheroid is preferably a viability of the tumor cell group in the core part of the spheroid.

When a distribution of one target drug in the core part of the spheroid is higher than that of the other target drug, or when the cell viability in the spheroid treated with the one target drug is lower than that in the spheroid treated with the other target drug, the method may further include determining that the one target drug has higher efficacy in cancer or tumor treatment than the other target drug. The distribution of a drug in the core part may be inversely proportional to the cell viability in the spheroid. Specifically, the higher the distribution of a drug in the core part, the better the drug is delivered by penetrating into the peripheral part, which may be based on the fact that the drug distributed in the core part reduces the viability of the tumor cell group existing in the core part.

In the method, when a distribution of the one target drug in the core part of the spheroid is not observed at all or when the cell viability in the spheroid or the cell viability of the tumor cell group in the core part is not significantly reduced with treatment of the one target drug, compared to that without treatment of the one target drug, it is obvious to those skilled in the art that the one target drug may be evaluated or screened to be a drug that is not suitable to be used as a cancer or tumor therapeutic agent.

Another aspect provides a method for screening a tumor treating material, comprising incubating the 3D tumor spheroid and a candidate material.

The tumor spheroid is the same as described above.

The method comprises incubating the 3D tumor spheroid and the candidate material.

The candidate material may mean a drug expected to prevent or treat a tumor or cancer. The candidate material may be an organic material or an inorganic material, and may be a single compound or a composite compound. In addition, the candidate material may be protein, peptide, DNA, RNA, etc., and all materials existing in nature may be used as candidate materials.

Next, the method may comprise measuring an expression level of a tumor biomarker of the cultured spheroid or determining whether cultured spheroid has survived or not.

The biomarker is a material capable of differentially diagnosing individuals in a normal group and individuals having a tumor or cancer group, and may include all of organic biomolecules increasing or decreasing in the individuals having a cancer, such as polypeptide, protein, nucleic acid, gene lipid, glycolipid, glycoprotein or sugar. A change in the expression of a tumor or cancer biomarker may be an increased or a decreased expression.

When the expression of a tumor biomarker of the cultured spheroid is increased or decreased, or when the cultured spheroid is killed, the method provides screening a candidate material.

Another aspect provides a method for providing information on cell permeability of an anticancer drug using the 3D tumor spheroid.

The spheroid and the caner are the same as described above.

In the present specification, the term “anticancer drug” collectively refers to a drug that kills a cancer cell while preventing division of the cancer cell. The anticancer drug has a feature of penetrating into a surface of a cancer cell. Therefore, the information on the cell permeability of the anticancer drug may be a clue in predicting the effect of the anticancer drug. If the anticancer drug is assessed to have a high cell permeability, it may be determined that the anticancer drug is effective in treating a cancer.

The anticancer drug may be vatalanib, lenvatinib, dovitinib, ammonium-glycyrrhizinate, epirubicin, temsirolimus, lintitript (SR-27897), cabozantinib, tesmilifene (DPPE), KX-01 (KX2-391, tirbanibulin), rubitecan, bardoxolone-methyl, mifepristone, mitomycin-c, genistein, paclitaxel, carboxyamidotriazole, pazopanib, halofuginone, vinblastine, SGX523, lonafarnib, marimastat, patupilone (epothilone-b), derenofylline, lorlatinib, OSI-930, everolimus, capecitabine, tamoxifen, rucaparib, alisertib, canertinib, altretamine, etoposide (7-ethyl-10-hydroxycamptothecin), roquinimex, aminoglutethimide, talazoparib, imatinib, quinestrol, alectinib, verubulin, or tipifarnib.

The method may further comprise: inducing a regression analysis model considering various chemical features of various anticancer drugs or interactions thereof.

The method may further comprise performing regression analysis using the regression analysis model.

In the present specification, the term “regression analysis” refers to a statistical technique for estimating effects of one or more independent parameters on dependent parameters. For example, in a case of regression analysis with one independent parameter, a regression line may be obtained by constructing a distribution of points at which the independent parameter and dependent parameters meet. The regression line may be represented by: Y=a+bX1+cX2. Designing a regression analysis model may include a stepwise regression based assay, and utilization of a forward process as a method for selecting parameters.

The regression analysis may be performed using, as input parameters, one or more chemical features selected from the group consisting of molecular weight (M.W) of drug, distribution coefficient (Log P), water solubility (Log S), acid dissociation equilibrium constant (pKa), physiological charge, hydrogen acceptor count, hydrogen donor count, polar surface area, rotatable bond count, polarizability, refractivity, and number of rings.

The regression analysis may use, as an output parameter, a difference in viability (or permeability) between a 3D multicellular tumor spheroid and a 3D single cell spheroid.

A model of the regression analysis may include a regression analysis equation represented by Equation 1, and

the regression analysis equation may be an equation for deriving a predicted cell viability difference (or permeability):

PO=a+bK+cL+dM+eN+fO+gP+hQ+iR+jS [Equation 1]

wherein PO is a predicted cell viability difference (or permeability), K is M·W(g/mol), L is log P, M is log S, N is a hydrogen acceptor count (units), O is a hydrogen donor count (units), P is a polar surface area (Å2), Q is a rotatable bond count (units), R is refractivity (m3/mol), S is polarizability (Å3), and a to j are constant values: a=0.1235313827, b=0.0035738171, c=0.0340283393, d=0.0340283393, e=−0.000519426, f=0.0108241714, g=−0.002123276, h=0.0007481956, i=−0.0050597, and j=−0.018713752.

The regression equation may be an equation in which a difference value in the cell viability (or permeability) between the 3D multicellular tumor spheroid and the 3D single cell spheroid are normalized between 0 and 1. The difference value being closer to 1 may mean that the permeability predicted by the chemical features is more similar to actual permeability. The multiple regression analysis may be a linear fitting method, and may be performed by deriving a regression line by identifying a distribution of points where permeability values predicted by chemical features and actual permeability values meet each other. A determinant coefficient (R²) of the regression line may be 0.60 to 0.75 or 0.62 to 0.70.

The method may further comprise: comparing the derived cell viability difference (or permeability) with an actual output parameter; and assessing permeability of the anticancer drug on the basis of the comparison result.

In another aspect, provided is a use of the 3D tumor spheroid or a composition comprising the same in manufacturing a cell model.

In another aspect, provided is a use of the tumor spheroid or a composition comprising the same in manufacturing a screening composition of a tumor therapeutic material.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 shows diagrams of three types of 3D multicellular tumor spheroids;

FIG. 2 shows a workflow used in one or more embodiments;

FIGS. 3 to 6 show analysis results of 3D multicellular tumor spheroids in terms of actual image, diameter, roundness and sphericity;

FIG. 7 shows observation results of three types of 3D multicellular tumor spheroids after fluorescence staining;

FIG. 8 shows SEM images of three types of 3D multicellular tumor spheroids, A, D and G being images of ASC+MDA-MB-231, B, E and H being images of BMSC+MDA-MB-231, and C, F and I being images of FIB+MDA-MB-231;

FIG. 9 shows immunofluorescence staining observation results of ECM markers for three types of 3D multicellular tumor spheroids;

FIGS. 10 to 13 show results of doxorubicin penetration into three types of 3D multicellular tumor spheroids, identified as, from the left, ASC+MDA-MB-231, BMSC+MDA-MB-231, and FIB+MDA-MB-231, and the doxorubicin penetration being represented in red in each figure;

FIG. 14 shows analysis results of cell viability in three types of 3D multicellular tumor spheroids when treating with 10 μM doxorubicin for 48 hours;

FIGS. 15 and 16 shows measurement results of cell apoptosis and necrosis in three types of 3D multicellular tumor spheroids when treating with 10 μM doxorubicin for 48 hours;

FIG. 17 shows analysis results of cell viability in a monolayer in a case where MDA-MB-231 2D breast cancer cell monolayer is treated with 10 μM doxorubicin co-cultured with fibroblast cells for 48 hours;

FIG. 18 shows observation results of ECM markers of 3D multicellular tumor spheroids having different co-cultured cell density ratios, the ASC:MDA-MB-231 co-culture ratios being 10:0, 3:7, 5:5, 7:3, and 0:10 from the left;

FIG. 19 shows results of doxorubicin penetration into 3D multicellular tumor spheroids having different co-cultured cell density ratios, the ASC:MDA-MB-231 co-culture density ratios being 10:0, 3:7, 5:5, 7:3, and 0:10 from the left, and the doxorubicin penetration being represented in red in each figure;

FIG. 20 shows analysis results of relative viability of cells in different types of 3D multicellular tumor spheroids, for each co-cultured cell density ratio, when treating with 10 μM doxorubicin for 48 hours;

FIGS. 21a and 21b shows names of 44 anticancer drugs used in drug efficacy evaluation, except for doxorubicin, and compound structures thereof;

FIG. 22 shows comparative analysis results of cell viability in a 3D multicellular tumor spheroid (ASC+MDA-MB-231) and a 3D single cellular tumor when treating with 44 anticancer drugs being in clinical use or clinical trial, except for doxorubicin, for 48 hours;

FIG. 23 shows results of comparing chemical features of 16 anticancer drugs for multiple regression analysis with cell viabilities in a 3D multicellular tumor spheroid (ASC+MDA-MB-231) and in 3D single cellular tumor spheroid;

FIG. 24 is a graphical representation of multiple regression analysis results, showing actual experimental result values and result values predicted by chemical features of the anticancer drugs shown in FIG. 23;

FIG. 25 shows analysis results of epirubicin penetration into three types of 3D multicellular tumor spheroids, the epirubicin penetration being represented in red in each figure;

FIG. 26 shows analysis results of topotecan penetration into three types of 3D multicellular tumor spheroids, the topotecan penetration being represented in green in each figure; and

FIG. 27 shows results of actual images obtained in cases where 3D single cellular tumor spheroids are formed using five types of solid tumor cells (A549, HT1080, MKN45, SK-BR-3, and MCF-7) (upper side), and 3D multicellular tumor spheroids are formed by co-culturing those solid tumor cells with ASC (lower side);

FIG. 28 shows observation results of cell distributions of 3D multicellular tumor spheroids derived from five types of solid tumor cells after fluorescence staining; and

FIG. 29 shows observation results of ECM markers of 3D single cellular tumor spheroids and 3D multicellular tumor spheroids, derived from three types of solid tumor cells after immunofluorescence staining.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the present disclosure will be described in further detail with reference to embodiments. However, the present disclosure is not limited to the disclosed embodiments.

Experimental Materials

1. Cell Lines

Human adipose-derived stromal cells (ASC) were purchased from Cefobio (Seoul) and were maintained in ASC growth culture media (Cefobio, Seoul, Korea) supplemented with 10% FBS, 1% L-glutamine and penicillin/streptomycin. The culture media were replaced every other day.

Bone marrow stromal cells (BMSC) and Human dermal fibroblast (FIB) were purchased from the Catholic University of Korea (Seoul, Korea) and were maintained in DMEM 1× culture media (Gibco™, Cat #11965092) supplemented with 10% FBS, 1% L-glutamine and penicillin/streptomycin. The culture media were replaced every other day.

MDA-MB-231 human breast cancer cell line, A529 human lung cancer cell line, HT1080 human fibrosarcoma cell line, MKN45 human stomach cancer cell line, SK-BR-3 human breast cancer cell line, and MCF-7 human breast cancer cell line were purchased from The Korean Cell Line Bank (KCLB, Seoul), and were maintained in RPMI 1640 culture media (Gibco™, Cat #11875-093) supplemented with 10% FBS, 1% L-glutamine and penicillin/streptomycin. The cell lines were cultured in an incubator being under 37° C. and 5% CO2 conditions. The culture media were replaced every other day.

2. Poly-HEMA

120 mg/mL of a poly-hydroxyethyl methacrylate (poly-HEMA) stock solution diluted with 95% ethanol was prepared and was turned over to then apply a vortex thereto, and, in order to obtain a working solution of poly-HEMA, 1 mL poly-HEMA stock solution was added to 23 mL of 95% ethanol to then adjust a final concentration to 5 mg/mL. A new working solution was prepared whenever a new plate was prepared.

3. Additional Information

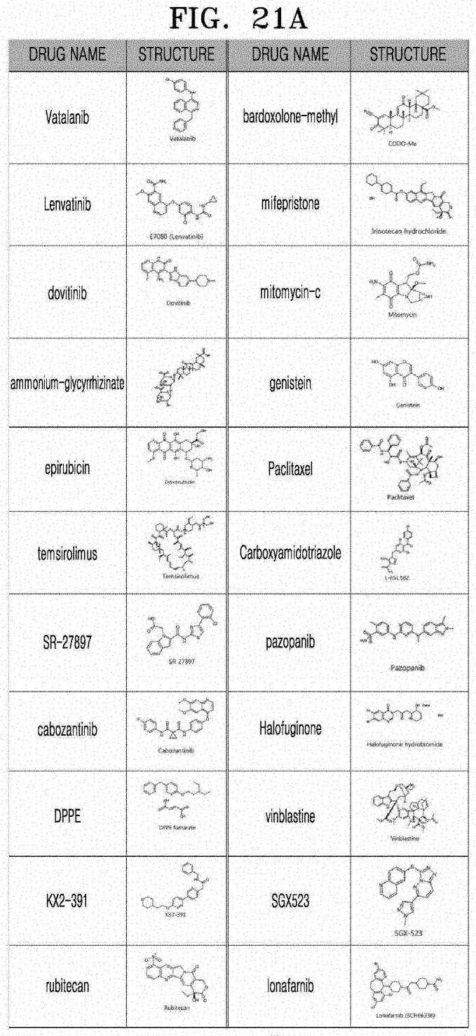

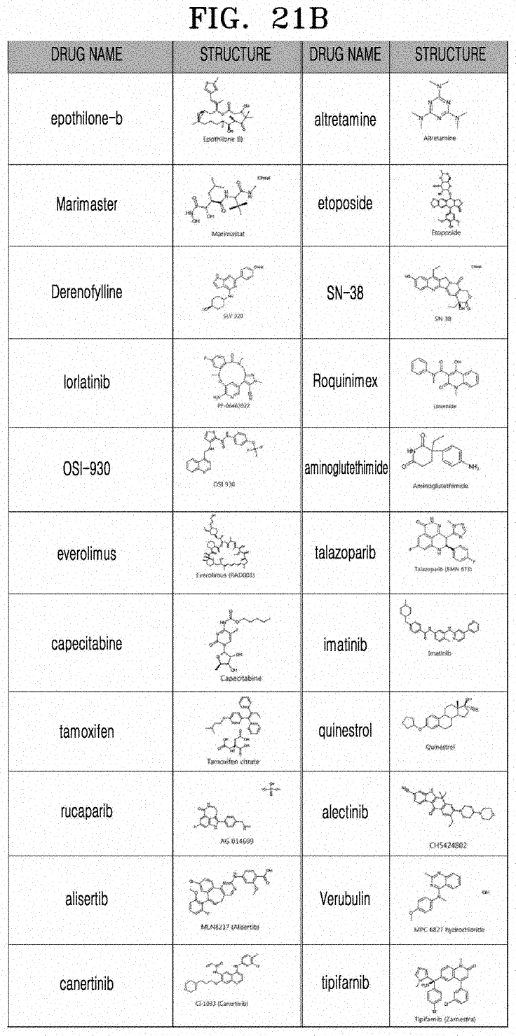

Matrigel (Matrigel® Basement Membrane Matrix, Corning, Cat #354234), anticancer drug (doxorubicin, SIGMA, #D1515), cell viability assay (Real Time-Glo™ MT Cell Viability Assay, Promega, Cat #G9711), and cell apoptosis/necrosis assay (Real Time-Glo™ Annexin V Apoptosis and Necrosis Assay, Promega, Cat #JA1011), were used. In addition, doxorubicin and 44 anticancer drugs, for drug efficacy evaluation, were supplied from Korea Chemical Bank of Korea Research Institute of Chemical Technology, and a list of the 44 kinds of anticancer drugs is shown in FIGS. 21a and 21b.

Experimental Method

1. Preparation of Non-Adsorptive Poly-HEMA Plate

60 μL of a poly-HEMA stock solution was pipetted to each well of a 96-well U-bottom plate and then evaporated in a 30° C. incubator with a lid for one week.

2. Formation of 3D Multicellular Tumor Spheroids

In order to form 3D multicellular tumor spheroids, suspensions of various cell types were prepared in growth culture media. Stromal cells were seeded to each well of a 96-well plate pre-coated with poly-HEMA at a density of 0.5×10⁴ cells/well, and then incubated in a 5% CO₂ incubator at 37° C. for 48 hours. Then, each 25 μL of tumor cells MDA-MB-231, A549, HT1080, MKN45, SK-BR-3, and MCF-7 was plated on the stromal cells in each well of the plate at a density of 0.5×10⁴ cells/well. After seeding the cells, the plate was centrifuged at 1000 rpm for 2 minutes to collect cells from the center of the well. 50 μL of a 10% matrigel solution diluted in the growth medium was gently added to a plate on ice to prevent matrigel from being gelled. Next, the plate containing cells and matrigel was centrifuged at 1000 rpm for 2 minutes. The plate was centrifuged at 1000 rpm for 2 minutes and then incubated in a 5% CO₂ incubator at 37° C. for 48 hours.

3. Morphological Analysis

The cells were seeded using a camera with a 5× objective lens attached to a microscope, and then images of all rotating ellipsoids were photographed for two days. All images were analyzed using software ImageJ (National Institutes of Health, Bethesda, Md., USA).

4. Scanning Electron Microscope (SEM) Analysis

After a two-day cell culture, 3D multicellular tumor spheroids were washed with PBS three times. To fix 3D spheroids, the 3D spheroids were treated with 2.5% glutaraldehyde at 4° C. for one hour and then fixed with 1% osmium tetroxide in deionized water for two hours. The fixed 3D spheroids were dehydrated twice (each 5 minutes) with a series of graded ethanols (30%, 50%, 70%, 80%, 90%, and 100%), and then treated with hexamethyldisilazane (HMDS) for two minutes, followed by vacuum drying overnight. Prior to use of a scanning electron microscope (SEM), the 3D spheroids were transferred to an adhesive carbon tape, and then sputter-coated with gold at 10 mA for 60 seconds. SEM images were photographed at 15 kV (Inspect F50).

5. Cell Labelling

The cells were labelled with chloromethyl fluorescein diacetate (CMFDA, molecular probe) as a cell tracker dye at room temperature for 30 minutes. To observe a distribution of co-cultured cells in 3D spheroids, before seeding on a plate, stromal cells and tumor cells were stained with a cell tracker green CMFDA dye and a cell tracker Red CMTPX dye. After forming 3D spheroids, the cells were observed using a confocal microscope (LSM700, Zeiss).

6. Immunofluorescence Staining

Section samples for immunofluorescence staining were washed with distilled water to remove OCT compounds, and were then allowed to pass through 0.25% triton X-100 in PBS at room temperature for 15 minutes. The samples were washed with PBS three times (5 minutes each time). After the samples were blocked with 3% bovine serum albumin (BSA) at room temperature for one hour, the samples were incubated with a mouse anti-collagen type I antibody (1:200), rabbit anti-fibronectin antibody (1:200) at 4° C. overnight. The samples were washed with PBS three times, and then incubated with Alexa Fluor® 488-donkey anti-mouse IgG (1:500) for one hour. The samples were washed with PBS and then observed using a confocal microscope.

7. Cell Viability Assay

For comparison of anticancer drug efficacy for 3D multicellular tumor spheroids, the spheroids were treated with 10 μg/mL drugs (doxorubicin, epirubicin, topotecan, or 44 types of anticancer drugs shown in FIG. 21) for two days. Each drug stock solution (1 mg/mL) was diluted in the culture medium so as to have a final concentration of 2× immediately before use. The spheroid in the culture medium (50 μL) formed by a two-day 3D cell culture was transferred to a multi-walled plate having opaque walls, ATP based cell viability assay was performed using Real Time-Glo™ MT cell viability assay (Promega, Cat #G9711). As solutions for Real Time-Glo™ MT cell viability assay (Promega, Cat #G9711), drug-containing solutions of the same volume and amount were added to each well. Thereafter, luminescence was recorded using a Glomax-Multi-Microplate reader (Promega, Glomax Discovery) for an integration time of 0.25-1.0 second per well.

8. Cell Apoptosis and Necrosis Assays

Apoptotic and necrotic cells in spheroids were assayed using Real Time-Glo™ Annexin V apoptosis and necrosis assays. Real Time-Glo™ Annexin V apoptosis and necrosis assays were performed in combination with a cell viability assay. The spheroid in the culture medium (50 μL) formed by a two-day 3D cell culture, were transferred to a multi-walled plate having opaque walls, and then treated with 50 μL of 2× drug (doxorubicin) until a total concentration reached 10 μM. Thereafter, 2X apoptosis and necrosis formulations of the same volume (100 μL) were added to each well according to standard protocol proposed in manufacturer's instructions. Luminescence and fluorescence values were measured using a Glomax-Multi-Microplate reader (Promega, Glomax Discovery) for an integration time of 0.25-1.0 second per well.

9. Treatment of Anticancer Drug

Doxorubicin, epirubicin, topotecan, or 44 types of anticancer drugs shown in FIG. 21 were diluted in 1:1 (RPMI-1640: DMEM) growth culture media, and 1000 μM/mL of a stock solution was prepared to then manufacture a working solution newly diluted every time drug treatment is performed.

10. Statistical Analysis

Statistical analysis of data was performed by an ANOVA one-way test using prism software (Graph Pad). Statistical significances were defined as *p<0.05, **p<0.01 and ***p<0.001. Multiple regression analysis was performed by stepwise regression using JMPpro statistical analysis software (JMP), and, for parameter selection, a forward process was utilized.

Experimental Results

1. Experimental Overview

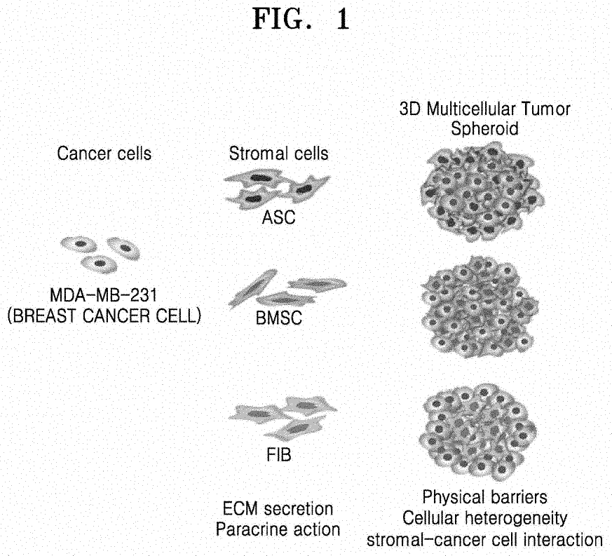

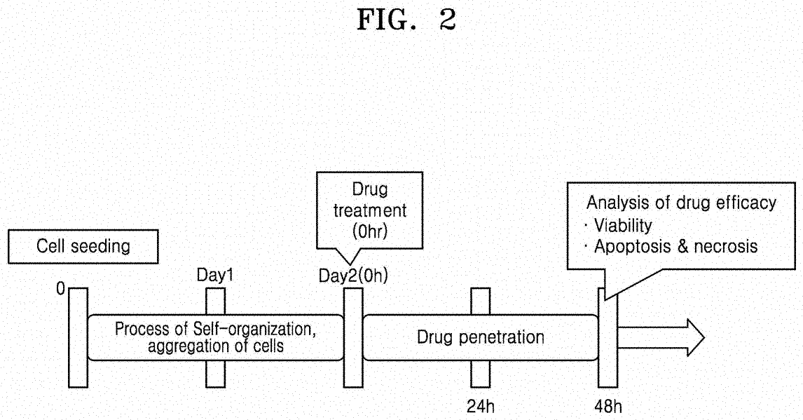

The experimental overview is as shown in FIGS. 1 and 2. Three types of multicellular tumor spheroids were produced using a co-culture system for two days (FIG. 1), and then inter-spheroids characteristics were analyzed. In addition, the drug penetration, cell viability and apoptosis rate of each of the multicellular tumor spheroids were measured, and corresponding data after two-day anticancer drug treatment were compared (FIG. 2).

2. Formation of 3D Multicellular Tumor Spheroids

(1) Co-Culture of Tumor Cells and Stromal Cells

To mimic a tumor microenvironment, tumor microenvironments for co-culturing three types of stromal cells and tumor cells were selected. Tumor cells on a 5% matrigel plate coated with poly-HEMA, and ASC, BMSC and FIB known as stromal cells in tumors, were co-cultured, yielding three types of multicellular tumor spheroids. During a 48-hour culture, 3D multicellular tumor spheroids were formed by self-organization of single cells on the plate.

(2) Diameters of 3D Multicellular Tumor Spheroids

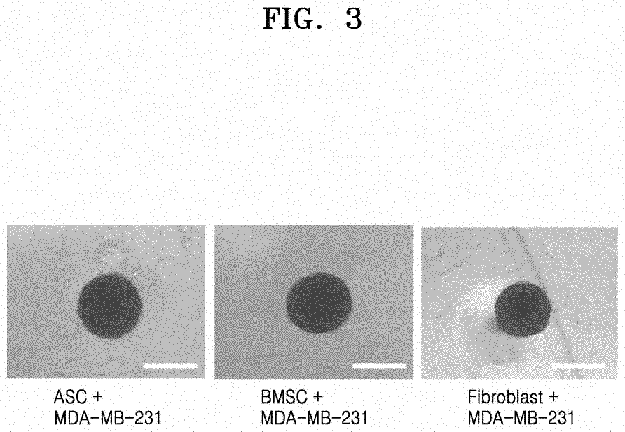

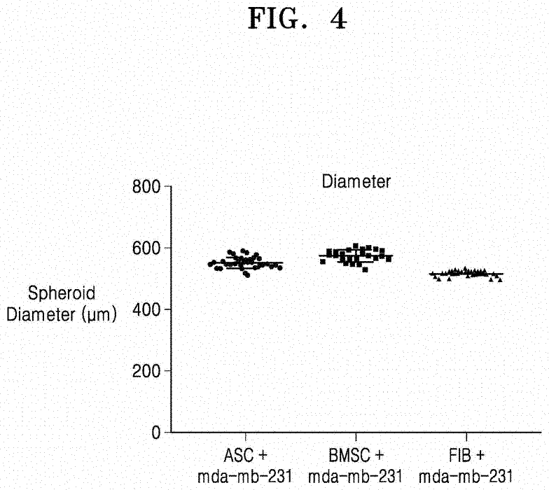

During the next two-day culture period, irrespective of types of stromal cells co-cultured with tumor cells, three types of 3D multicellular tumor spheroids having a diameter of 500 μm to 600 μm were formed, (FIGS. 3 and 4). The diameter in this range is an ideal model size for mimicking in vivo conditions with tumor spheroids having a diameter of 500 μm or greater. When the spheroid has the diameter in the above range, a physico-chemical gradient similar to micro metastases is demonstrated, indicating cells having a different proliferation stage from avascular tumor cells having a hypoxic core. For these reasons, by having the diameter of 500 μm to 600 μm, the three tumor spheroids are quite suitably used as in vitro 3D models for mimicking in vivo tumors.

(3) Roundness of 3D Multicellular Tumor Spheroids

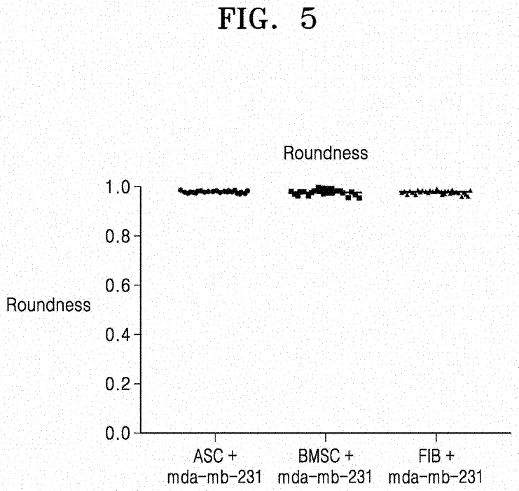

The roundness represents a clear or smooth boundary (2D). The extent of roundness of a spheroid indicates a roundness of a projected region of the spheroid. The roundness is in a range of 0 to 1, and the closer to 1 the roundness is, the higher the roundness of a projected region of the spheroid is. The produced three types of spheroids have a roundness of 0.99 or greater, which is close to 1.0 (FIGS. 3 and 5).

(4) Sphericity of 3D Multicellular Tumor Spheroids

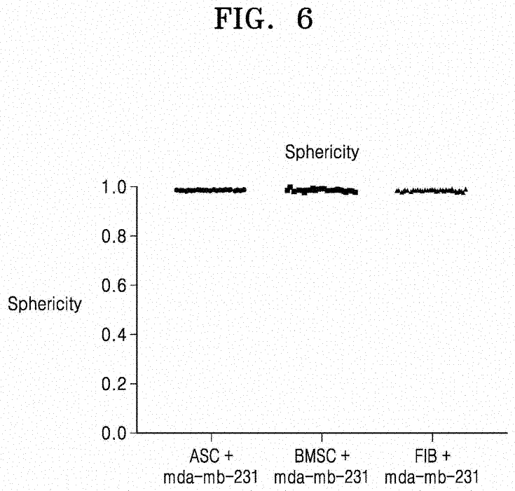

According to the sphericity, tumor spheroids may be classified as spherical tumor spheroids having a sphericity of 0.90 or greater (sphericity index; SI≥0.90) or non-spherical tumor spheroids having a sphericity of not greater than 0.90 (SI≤0.90). All of the produced three types of spheroids had a specificity index of greater than 0.99, that is, nearly close to 1.0. This suggests that all of the spheroids are formed in a spherical shape, that is, a three-dimensional well-defined shape (FIGS. 3 and 6).

3. 3D Multicellular Tumor Spheroid Assay

(1) Distribution of Tumor Cells in 3D Multicellular Tumor Spheroids

In order to mimic an interaction between a stromal cell and a cancer (tumor) cell, which is one of tumor microenvironment characteristics, 3D multicellular tumor spheroids were designed. Distributions of positions of the respective cell types (breast cancer cells and stromal cells) were identified from the 3D multicellular tumor spheroids using a cell tracker. Before seeding, stromal cells (ASC, BMSC and FIB) and human breast cancer cells (MDA-MB-231) were stained with a Cell Tracker™ Green CMFDA dye and a Cell Tracker™ Red CMTPX dye. After forming 3D multicellular tumor spheroids, distributions of cancer cells were observed in the spheroids using a confocal microscope.

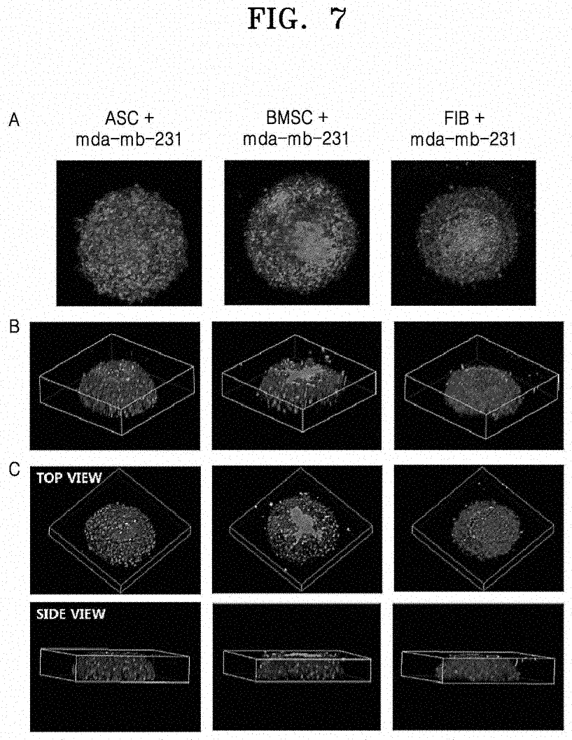

Interestingly, the cancer cells and stromal cells were differently distributed in the 3D multicellular tumor spheroids according to the type of stromal cell (FIG. 7). The ASC+MDA-MB-231 spheroid looked as if it were covered with ASC as a breast stromal cell, and MDA-MB-231 human breast cancer cells were positioned inside the spheroid. In contrast, FIB+MDA-MB-231 spheroids were observed such that most of MDA-MB-231 human breast cancer cells were positioned at exterior regions of the spheroids, as compared with the co-cultured fibroblast cells. The BMSC+MDA-MB-231 spheroid had human breast cancer cells and stromal cells uniformly distributed therein.

(2) Surfaces of 3D Multicellular Tumor Spheroids

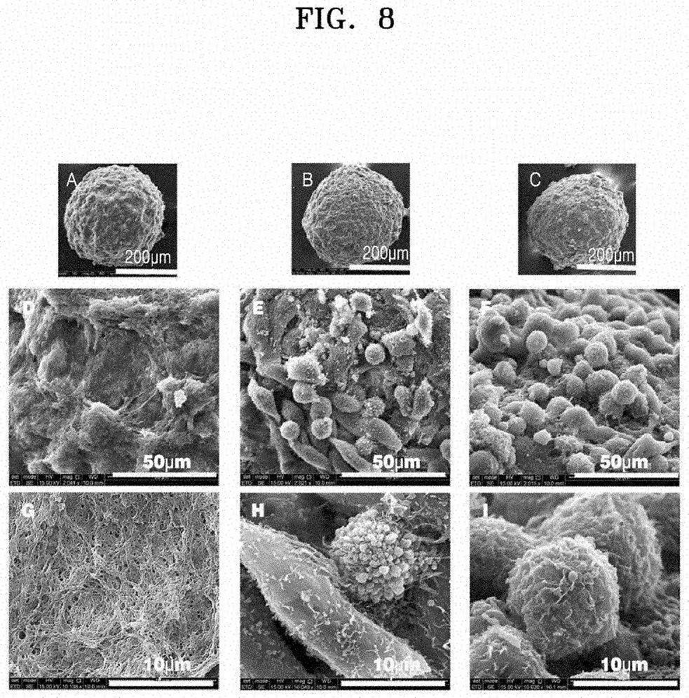

Surfaces of three types of 3D multicellular tumor spheroids were observed and compared, and, as a result, all of the three types of 3D multicellular tumor spheroids had surprisingly different surfaces according to the type of stromal cell (FIG. 8).

The ASC+MDA-MB-231 spheroid looked as if it were surrounded by ECM components and had a smooth surface without any kind of cell found. Meanwhile, much more cancer cells were distributed on the surface of the FIB+MDA-MB-231 spheroid than on the surfaces of other spheroids, and cancer cells and stromal cells were uniformly distributed on the surface of the BMSC+MDA-MB-231 spheroid.

(3) Accumulation of ECMs on 3D Multicellular Tumor Spheroids

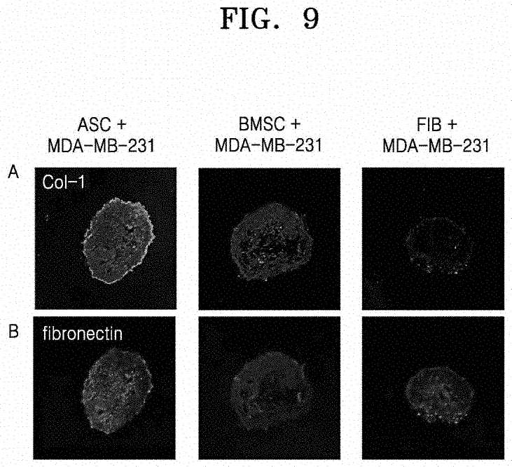

3D multicellular tumor spheroid sections were immunofluorescence stained on extracellular matrix (ECM) protein, collagen type 1 and fibronectin. Type 1 collagen and fibronectin were much more abundantly expressed in the ASC+MDA-MB-231 spheroid than the FIB+MDA-MB-231 spheroid and the BMSC+MDA-MB-231 spheroid (FIG. 9).

Specifically, collagen type 1 was over-expressed on the surface of the ASC+MDA-MB-231 spheroid, compared to other regions of the ASC+MDA-MB-231 spheroid. This reflects a fact that collagen is an ECM protein present most abundantly on areas other than a primary breast cancer area. In addition, the smooth property of the ASC+MDA-MB-231 spheroid surface due to high ECM secretion may contributes to hiding shapes of single cancer cells on most parts of the spheroid surface.

As confirmed from the ASC+MDA-MB-231 spheroid, over-expression of ECMs is one of important factors in replication of a cancer tissue, and a cell-ECM interaction plays a key role in a tumor microenvironment. Specifically, expression levels of collagen type 1 and fibronectin were increased in breast cancer cells, which are associated with growth, metastasis and progression of a tumor. In addition, ECM components in the tumor microenvironment and over-expression of collagen type 1 and fibronectin play major roles in drug resistance as well as cancer progression. Particularly, the collagen type 1 is a drug resistance increasing factor. For these reasons, responses to the anticancer drugs may be predicted to be all different due to a noticeable difference in the expression level of ECM proteins in the three 3D multicellular tumor spheroids.

(4) Drug Penetration into 3D Multicellular Tumor Spheroids

To assess drug penetration into three types of 3D multicellular tumor spheroids, the spheroids were treated with doxorubicin as a chemotherapeutic formulation for two days. Doxorubicin is one of common chemotherapeutic agents used in treating a variety types of tumors. To confirm whether structural characteristics have impacts on anticancer drug penetration into spheroids, it was analyzed whether anticancer drug resistance, e.g., insufficient penetration of anticancer drug into the spheroids, was observed using natural red fluorescence of doxorubicin. Fluorescent images after the spheroids were treated with doxorubicin for 48 hours confirmed differential distributions of drugs according to the type of spheroid.

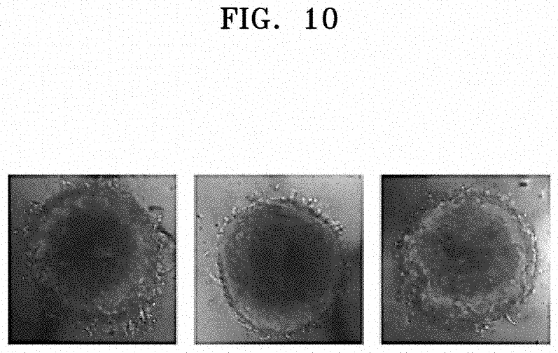

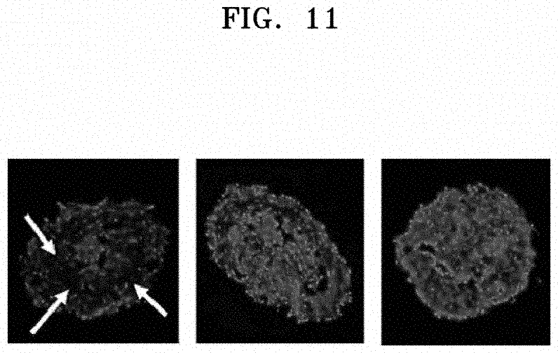

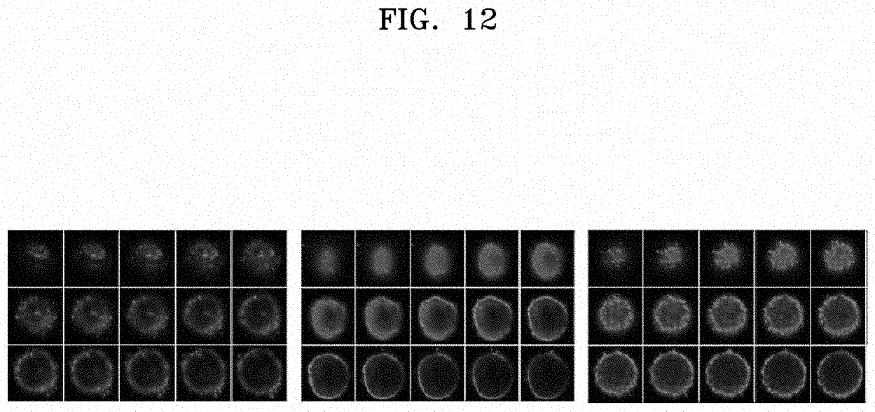

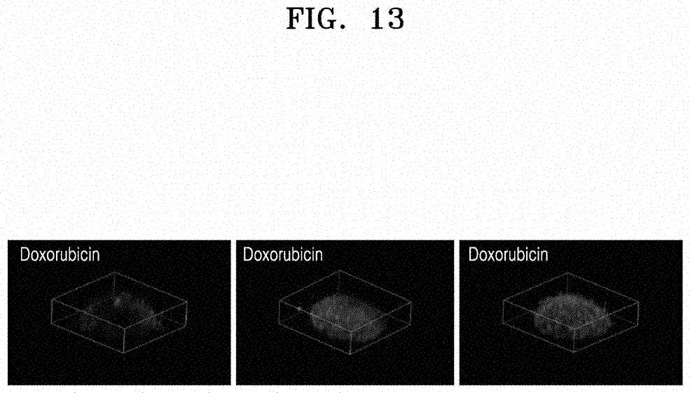

Confocal images of doxorubicin showed differences in the extent of doxorubicin penetrating into the spheroids (FIGS. 10 to 13). Little distribution of doxorubicin was observed at points indicated by white arrows in FIG. 11, confirming that much less penetration of doxorubicin into the ASC+MDA-MB-231 spheroid, the surface of which is surrounded by ECM collagen type 1, than into other spheroids.

(5) Drug responses to 3D Multicellular Tumor Spheroids

1) Cell viability of 3D Multicellular Tumor Spheroids

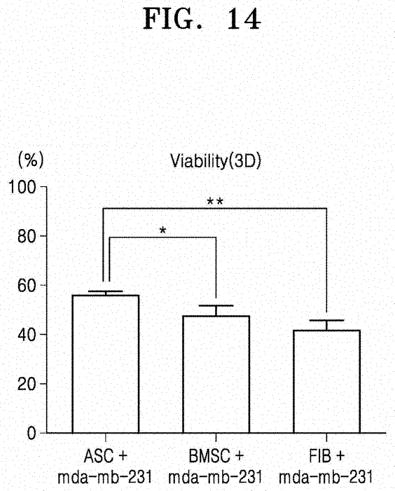

With regard to a low penetration level of the drug, the ASC+MDA-MB-231 spheroid treated with 10 μM doxorubicin for two days showed highest cell viability (56.67%), as compared with other types of spheroids. The cell viability of the BMSC+MDA-MB-231 spheroid was 48.33%, which is lower than that of the treated ASC+MDA-MB-231 spheroid, and the FIB+MDA-MB-231 spheroid observed to have a highest drug penetration level had a lowest cell viability (43%). There is a substantial difference in the cell viability between the ASC+MDA-MB-231 spheroid and the FIB+MDA-MB-231 spheroid (P=0.0024). These results suggest that the low level of a drug penetrating into a multicellular spheroid affects low drug efficacy or high drug resistance due to high viability (FIG. 14).

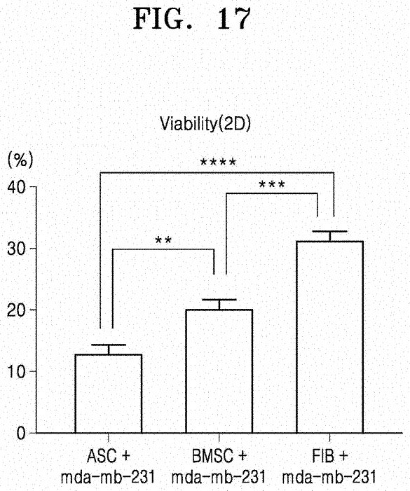

In addition, when the same experiment was carried out on a 2D culture plate, the MDA-MB-231 co-cultured with fibroblast cells demonstrated a highest cell viability (FIG. 17), while the lowest cell viability was observed in the MDA-MB-231 co-cultured with ASC.

In 3D versus 2D comparison of cell viability, the three types of 3D spheroids were all assessed to have lower drug sensitivity than 2D monolayer spheroids. In addition, doxorubicin responses in three types of 3D breast cancer models showed a propensity opposite to that of 2D cell models, confirming that the structural characteristics of the 3D tumor models in combination with differences in the drug efficacy for the 3D breast cancer models caused drug responses completely different from that for the 2D models.

This result presents a significance in the structural characteristic of a 3D tumor model, suggesting that a structural effect exerted by a 3D cell model, not by a 2D cell model, for screening the efficacy of an anticancer drug, may change the efficacy and propensity of drug.

2) Apoptosis or Necrosis of 3D Multicellular Tumor Spheroids

To investigate drug sensitivity depending on the viability against doxorubicin in the spheroids, Real Time-Glo™ Annexin V apoptosis and necrosis assays were performed. If apoptosis is induced by a drug response, a cell membrane is reversed to expose phosphatidyl-serine (PS), and an apoptotic cell can be analyzed by binding PS with Annexin V detected by a luminescence signal. A necrotic cell is detected by binding DNA with PI (green) that produces a fluorescence signal when the necrotic cell invades into a cell and a cell membrane loses integrity.

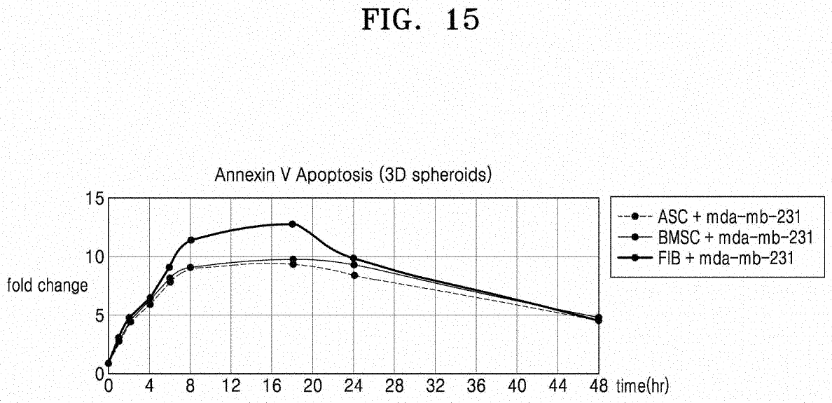

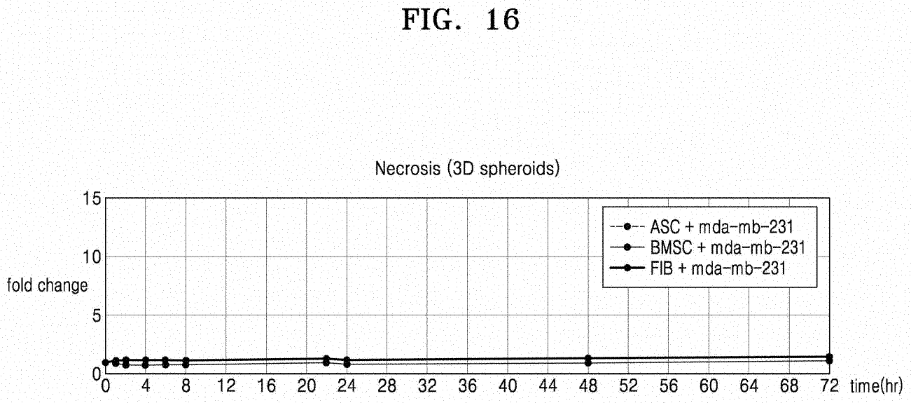

A fold change in the expression of Annexin V as an initial apoptosis marker correlates with viability. A highest increasing level of annexin V was observed from the FIB+MDA-MB-231 spheroid treated with doxorubicin. In addition, a lowest level of annexin V was observed from the ASC+MDA-MB-231 spheroid treated with doxorubicin. As confirmed from FIG. 15, the apoptosis in the FIB+MDA-MB-231 spheroid treated with doxorubicin was increased about 13 times that in the BMSC+MDA-MB-231 spheroid (10-fold) and the ASC+MDA-MB-231 spheroid (9-fold). In addition, a difference in the doxorubicin-induced apoptosis between spheroid types was reflected on the cell viability of each spheroid. No significant difference in the necrosis (PI) was observed from any type of the 3D multicellular tumor spheroids (FIG. 16).

4. Formation of 3D Multicellular Tumor Spheroids depending on Change in Conditions and Assays Thereof

(1) Change in Co-Culture Ratios

1) Accumulation of ECMs

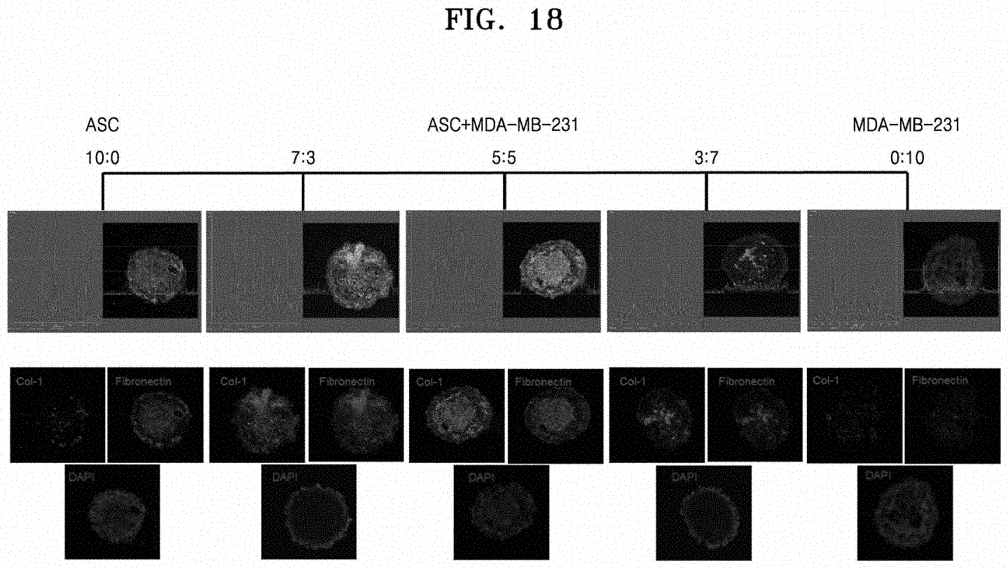

Expression of extracellular matrix (ECM) protein in 3D multicellular tumor spheroids according to the co-culture ratio of ASC as a stromal cell and MDA-MB-231 as a breast cancer cell was investigated. 3D multicellular tumor spheroids were formed by varying co-culture ratios of ASC:MDA-MB-231 to 10:0, 3:7, 5:5, 7:3 and 0:10, sections of the 3D multicellular tumor spheroids were immunofluorescence stained to collagen type 1 and fibronectin. As a result, when the co-culture ratio of ASC:MDA-MB-231 was 5:5, expression levels of collagen type 1 and fibronectin were highest (FIG. 18).

2) Drug Permeation and Cell Viability

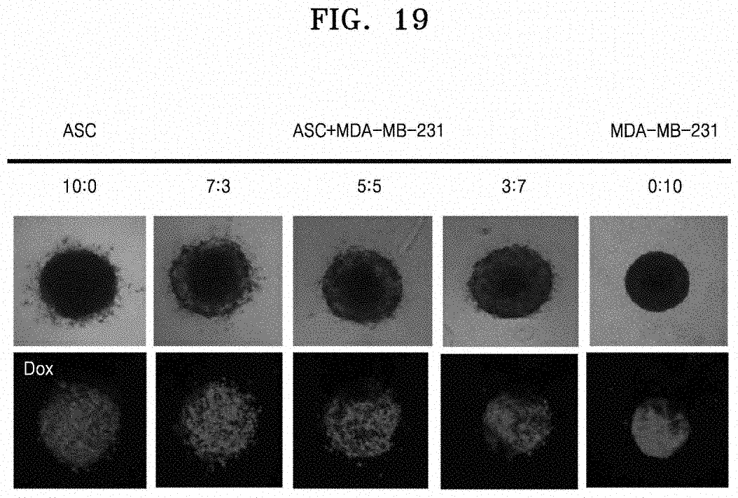

To assess drug penetration into 3D multicellular tumor spheroids depending on the co-culture ratio, the spheroids were treated with doxorubicin as a chemotherapeutic agent for two days. Thereafter, actual images of the spheroids were observed, and the extent of a drug penetrating into the spheroids was assessed using natural red fluorescence of doxorubicin. As a result, fluorescent images of spheroids after the spheroids are co-cultured with doxorubicin for 48 hours showed differential drug distributions according to co-culture ratios. Specifically, the 3D multicellular tumor spheroid having a co-culture ratio of ASC:MDA-MB-231 being 5:5, was confirmed to have a drug less penetrating into the core thereof than other spheroids having different co-culture ratios (FIG. 19).

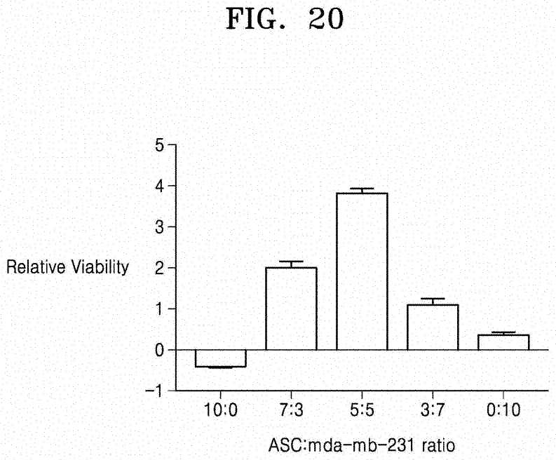

Next, the viability of each of 3D multicellular tumor spheroids depending on the co-culture ratio was analyzed. As a result, the 3D multicellular tumor spheroid having a co-culture ratio of ASC:MDA-MB-231 being 5:5 was confirmed to show a relatively high viability compared to other spheroids having different co-culture ratios (FIG. 20).

The results showed that the expression level of ECM was higher in the 3D multicellular tumor spheroid having stromal cells and breast cancer cells co-cultured in a 5:5 (1:1) ratio than in other spheroids having different co-culture ratios, suggesting that penetration of doxorubicin into the spheroid was inhibited by the high expression level of ECM. In addition, the viability was highest in the 3D multicellular tumor spheroid having the ASC:MDA-MB-231 co-culture ratio of 5:5, in which doxorubicin penetration was least, and thus drug penetration into the spheroid was highly inhibited, suggesting that the inhibited drug penetration may affect low efficacy or high resistance of drug.

(2) Change of Anticancer Drug

1) Cell Viability

To confirm drug penetration inhibiting effects of 3D multicellular tumor spheroids for various anticancer drugs, 44 anticancer drugs being in clinical use or clinical trial, except for doxorubicin, were treated for 48 hours, and viabilities of cells in the 3D multicellular tumor spheroid (ASC+MDA-MB-231) and the 3D single cellular tumor spheroid were comparatively analyzed (FIG. 21). If the viability in the 3D multicellular tumor spheroid is higher than that in the 3D single cell tumor spheroid, this may suggest that the 3D multicellular tumor spheroid would possibly have low drug efficacy or high drug resistance. In addition, it can be analogized that the high drug resistance may be affected by the extent of drug penetrating into a spheroid.

The 3D single cell tumor spheroid was formed by a single culture of tumor cells on a 5% matrigel plate coated with poly-HEMA. 50 μL tumor cells were plated on each well of the plate at a density of 0.5×10⁴ cells/well and then incubated in a 5% CO2 incubator at 37° C. for 48 hours. During a 48 hour culture, the 3D single cell tumor spheroid was formed by self-organization of the cells on the plate.

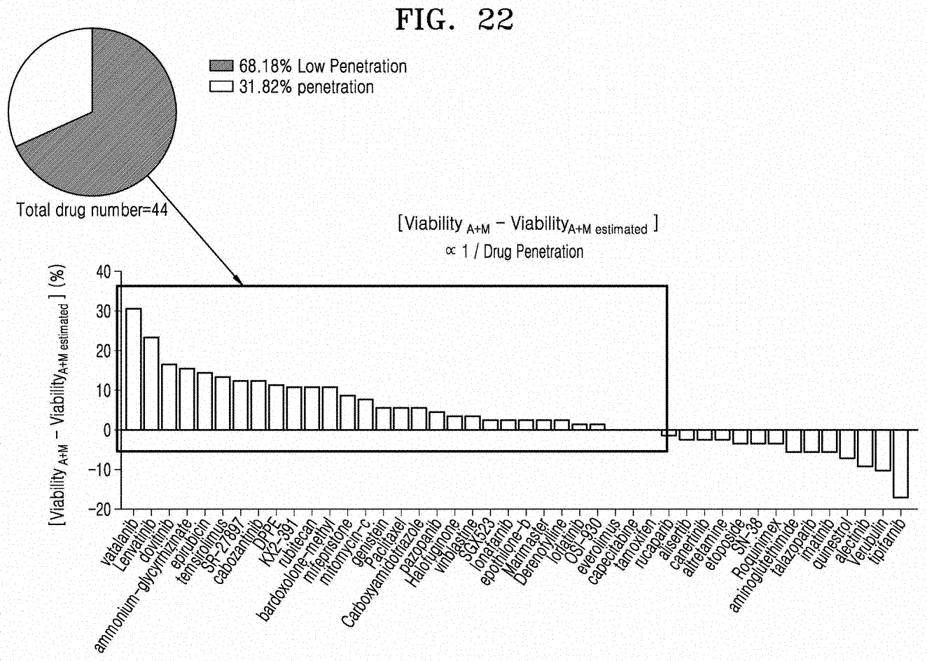

When 30 out of a total of 44 types of anticancer drugs (about 68.18%) were treated, the 3D multicellular tumor spheroids showed a higher viability than the 3D single cell tumor spheroid, and when 14 anticancer drugs (about 31.82%) were treated, the 3D multicellular tumor spheroids showed a lower viability than the 3D single cell tumor spheroid (FIG. 22). These results implicate that a high proportion of the anticancer drugs currently being used in clinical stages or pre-clinical stages may demonstrate high drug resistance in the 3D multicellular tumor spheroids. Further, the results also present a possibility of a 3D multicellular tumor spheroid being usable as an in vitro platform for observing permeability of newly developed anticancer drugs.

2) Confirmation of Relevance of Anticancer Drug with Chemical Features and Cell Viability

To confirm which of major chemical features of an anticancer drug affects viability in a tumor spheroid and drug penetration, a multiple regression analysis method was performed. In detail, as shown in FIG. 21, 16 anticancer drugs acting in a nucleus to thus demonstrate similar mechanisms, were deduced from among the 44 anticancer drugs. Next, chemical features of anticancer drugs were extracted from Drugbank database (https://go.drugbank.com/drugs).

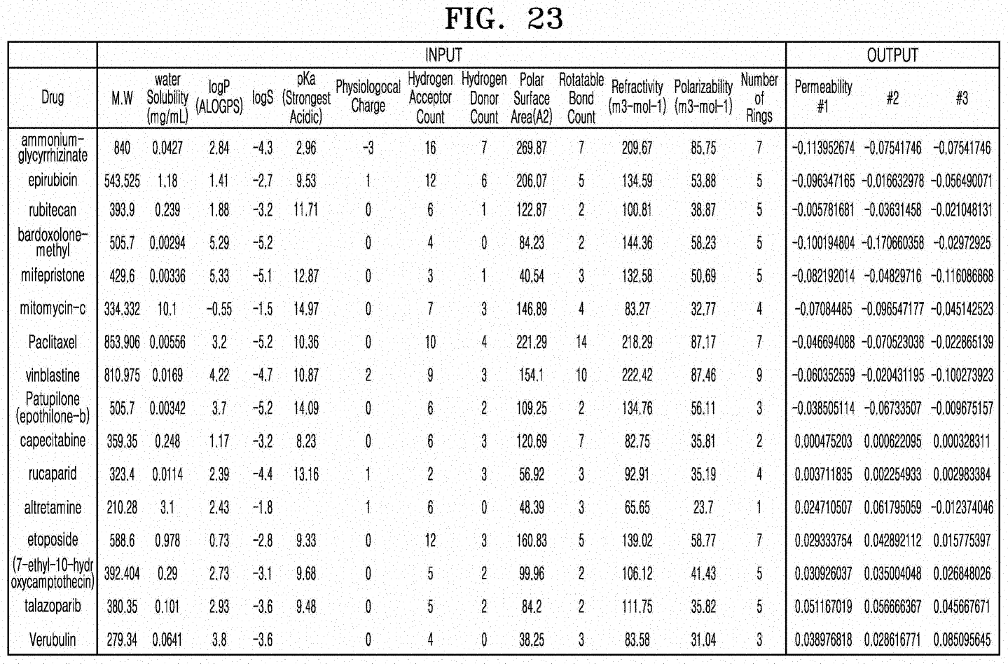

The extracted chemical features may include molecular weight (M·W) of drug, distribution coefficient (Log P), water solubility (Log S), acid dissociation equilibrium constant (pKa), physiological charge, hydrogen acceptor count, hydrogen donor count, polar surface area, rotatable bond count, polarizability, refractivity, and number of rings, which were used as input parameters (FIG. 23).

Thereafter, a difference in the viability (or permeability) between a 3D multicellular tumor spheroid and a 3D single cell spheroid was obtained from three independent experiments, and the obtained difference was used as an output parameter. Since a difference in individual chemical features of anticancer drugs could not perfectly account for cell viability, a multiple regression equation was derived in consideration of interactions among various factors as well as the respective chemical features.

PO=a+bK+cL+dM+eN+fO+gP+hQ+iR+jS

In the above equation, PO is a predicted cell viability difference (or permeability), K is M·W(g/mol), L is log P, M is log S, N is a hydrogen acceptor count (units), O is a hydrogen donor count (units), P is a polar surface area (Å2), Q is a rotatable bond count (units), R is refractivity (m3/mol), S is polarizability (Å3), and a to j are constant values: a=0.1235313827, b=0.0035738171, c=0.0340283393, d=0.0340283393, e=−0.000519426, f=0.0108241714, g=−0.002123276, h=0.0007481956, i=−0.0050597, and j=−0.018713752.

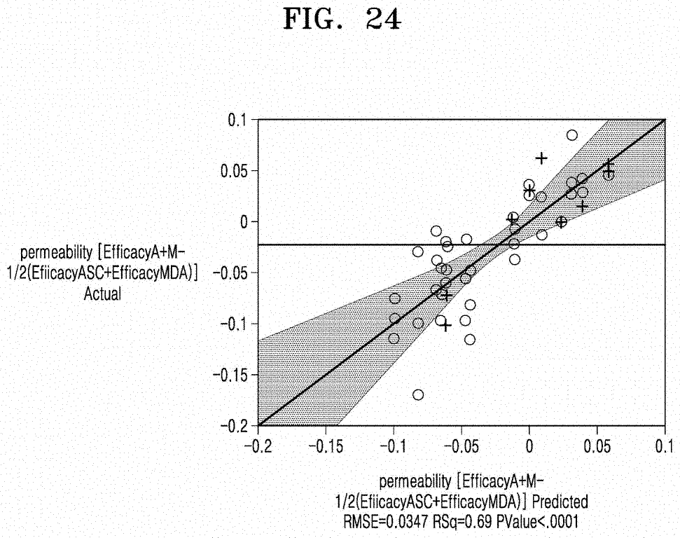

The present inventors compared the derived equation with the actual output parameter shown in FIG. 23. As a result, it was observed that statistically significant regression analysis was performed (p value <0.05). In addition, it was confirmed that a determinant coefficient was enough for input parameters and combinations thereof to account for the output parameter (R2=0.69) (FIG. 24). It is known that permeability of a compound is positively associated with Log P and is negatively associated with such chemical features as polar surface area, refractivity, or polarizability. The results signify that the anticancer drug permeability predicted through this regression analysis using 3D multicellular spheroids well satisfies a known correlation (c is a positive constant, and g, i and j are each a negative constant). The results also signify that a 3D multicellular spheroid model is a model that may well reflect permeability differences depending on chemical features of various anticancer drugs.

3) Penetration of Drug

Next, the extents of various types of anticancer drugs penetrating into 3D multicellular tumor spheroids were investigated. To this end, two chemo therapeutic agents, i.e., epirubicin and topotecan, used as therapeutic agents for various types of solid tumors, including a breast cancer, were used. Since epirubicin and topotecan naturally emit red fluorescence and green fluorescence, respectively, drug penetration can be detected by observing representation of fluorescence. Like in the previous case of doxorubicin, the FIB+MDA-MB-231 spheroid and the BMSC+MDA-MB-231 spheroid were used as control groups to be compared with the 3D multicellular tumor spheroid (ASC+MDA-MB231) in view of the extent of drug penetration.

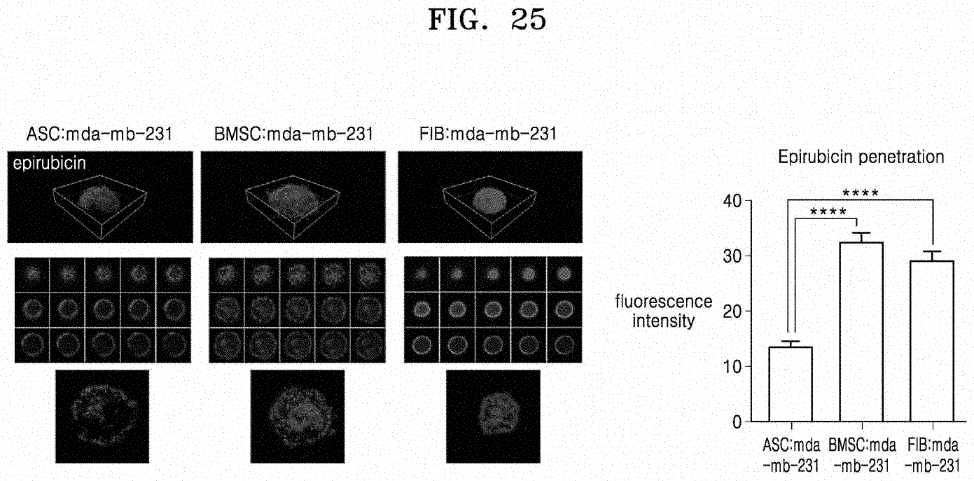

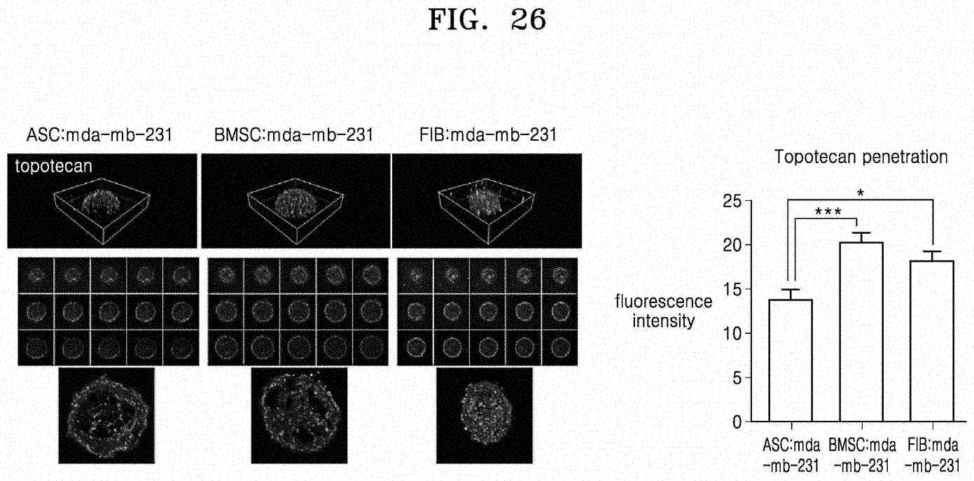

These anticancer drugs were treated in the spheroids for 48 hours, and distributions of the anticancer drugs were analyzed using a fluorescence microscope. As a result, in both of the anticancer drugs, the 3D multicellular tumor spheroid (ASC+MDA-MB-231) showed statistically signifixcantly low drug permeability, compared to BMSC+MDA-MB-231 and FIB+MDA-MB-231 spheroid (FIGS. 25 and 26). This result implies that 3D multicellular tumor spheroids are actually capable of inhibiting penetration of not only doxorubicin but also two additional anticancer drugs that are currently used as chemotherapeutic agents.

(3) Diversification of Tumor Cell

1) Distribution and Morphology of Tumor Cell

A solid tumor has a feature in that stromal cells are positioned on the surface of a tumor cell in a tumor microenvironment and ECMs are highly distributed on the surface of the tumor cell Therefore, an attempt was made to confirm whether such a feature is exhibited even in cases where 3D multicellular tumor spheroids are formed using solid tumor cells other than MDA-MB-231 breast cancer cells. To this end, A549 (lung cancer cell), HT1080 (fibrosarcoma cell), MKN45 (stomach cancer cell), SK-BR-3 (breast cancer cell) and MCF-7 (breast cancer cell) were used, and distributions of positions of the respective cell types ((solid tumor cells and stromal cells) were identified from the 3D multicellular tumor spheroids using a cell tracker. Before seeding, the stromal cells and solid tumor cells (A549, HT1080, MKN45, SK-BR-3, and MCF-7) were stained with a Cell Tracker™ Green CMFDA dye and a Cell Tracker™ Red CMTPX dye, respectively. After forming the 3D multicellular tumor spheroids, spheroid morphologies were observed using an optical microscope and distributions of cancer cells in the spheroids were observed in the spheroids using a confocal microscope.

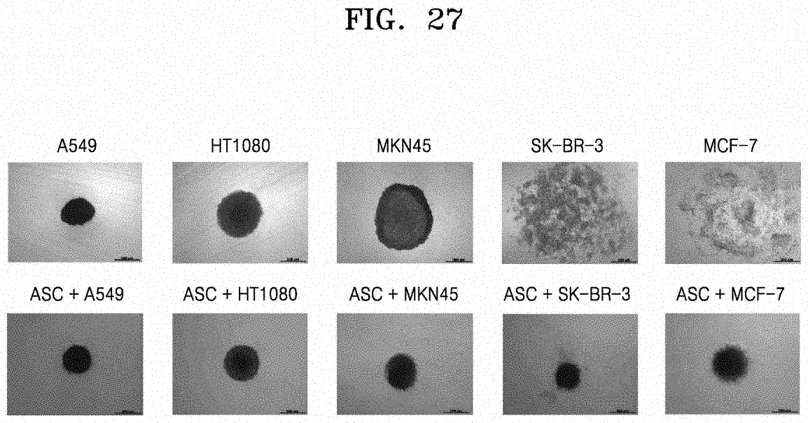

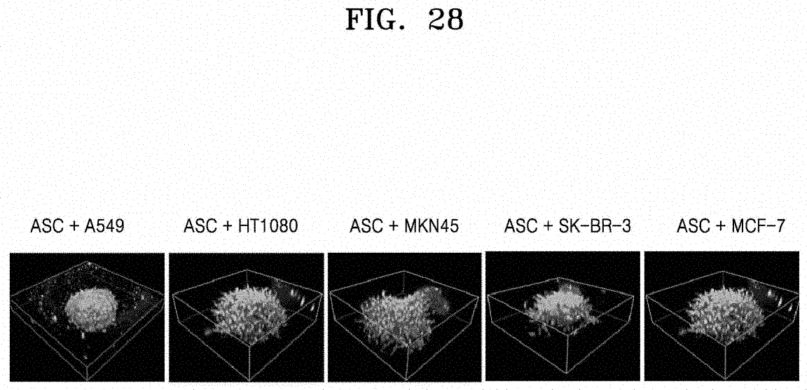

When 3D tumor spheroids were formed using single tumor cells, no spheroids were formed in most of solid tumor cells, or spheroids, if any, were formed in an inconsistent shape. However, in cases of 3D multicellular tumor spheroids, spheroids having a consistently spherical shape were formed in all of five types of solid tumor cells (FIG. 27). When distributions of cancer cells and stromal cells in the spheroids were observed, the cancer cells were positioned at interior regions, and stromal cells were at exterior regions of all the 3D multicellular tumor spheroids formed using five types of solid tumor cells (A549, HT1080, MKN45, SK-BR-3, and MCF-7) and stromal cells (FIG. 28).

2) Expression of ECM

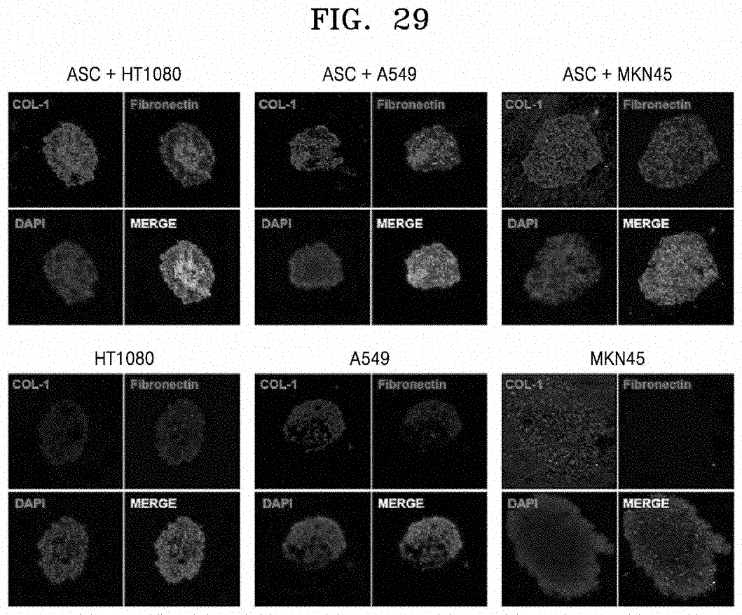

Next, in the 3D multicellular tumor spheroids using solid tumors, expression levels of extracellular matrix (ECM) protein, collagen type 1 and fibronectin were identified by immunofluorescence staining. Three types of solid tumor cells (HT-1080, A549, and MKN45) were used. Whereas there were little expression levels of collagen type 1 and fibronectin in the 3D tumor spheroids using single tumor cells, collagen type 1 and fibronectin were highly expressed in all the 3D multicellular tumor spheroids using three types of solid tumor cells, compared to single cell spheroids (FIG. 29). These results suggest that the 3D multicellular tumor spheroids formed using various types of solid tumor cells also appropriately reflect features of microenvironment of solid tumors (distributions of stromal cells positioned at peripheral portion and ECM positioned on spheroid surfaces).

The spherical 3D tumor spheroid according to an aspect has an appropriate diameter, roundness and specificity so as to be suitably used in vitro, and expresses an ECM structure similar to that of in vivo tumor, and thus may be used in evaluating the efficacy of drug for treating various types of tumors.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

1. A spherical 3D tumor spheroid comprising:
a core part including a tumor cell group; and
a peripheral part including an adipose-derived stromal cell group and an extracellular matrix component and surrounding the core part.

2. The spheroid of claim 1, wherein the tumor cell group comprises a breast cancer cell, a lung cancer cell, a fibrosarcoma cell, or a stomach cancer cell.

3. The spheroid of claim 1, wherein the tumor cell group comprises MDA-MB-231, A549, HT1080, MKN45, SK-BR-3 or MCF-7 cells.

4. The spheroid of claim 1, wherein the extracellular matrix component is a product expressed by an interaction between the tumor cell group and the adipose-derived stromal cell group.

5. The spheroid of claim 1, wherein the extracellular matrix component comprises collagen and fibronectin.

6. The spheroid of claim 1, wherein the spheroid has a diameter of 500 μm to 600 μm.

7. The spheroid of claim 1, wherein the spheroid has a roundness of 0.90 or greater and a sphericity of 0.90 or greater.

8. The spheroid of claim 1, wherein the spheroid is formed by co-culturing the tumor cell group and the adipose-derived stromal cell group at a cell density ratio of 7:3 to 3:7.

9. A method for evaluating efficacy of a cancer or tumor therapeutic agent comprising: treating target drugs to the spheroid of claim 1; and
analyzing distributions of the target drugs in a core part of the spheroid or analyzing cell viability in the spheroid.

10. The method of claim 9, further comprising determining that one target drug has higher efficacy in cancer or tumor treatment than the other target drug, when a distribution of the one target drug in the core part of the spheroid is higher than that of the other target drug, or when the cell viability in the spheroid treated with the one target drug is lower than that in the spheroid treated with the other target drug.

11. The method of claim 9, wherein the cell viability is a viability of a tumor cell group in the core part of the spheroid.

12. A method for evaluating efficacy of a cancer or tumor therapeutic agent comprising:
treating target drugs to the spheroid of claim 2; and
analyzing distributions of the target drugs in a core part of the spheroid or analyzing cell viability in the spheroid.

13. A method for evaluating efficacy of a cancer or tumor therapeutic agent comprising:
treating target drugs to the spheroid of claim 3; and
analyzing distributions of the target drugs in a core part of the spheroid or analyzing cell viability in the spheroid.

14. A method for evaluating efficacy of a cancer or tumor therapeutic agent comprising:
treating target drugs to the spheroid of claim 4; and
analyzing distributions of the target drugs in a core part of the spheroid or analyzing cell viability in the spheroid.

15. A method for evaluating efficacy of a cancer or tumor therapeutic agent comprising:
treating target drugs to the spheroid of claim 5; and
analyzing distributions of the target drugs in a core part of the spheroid or analyzing cell viability in the spheroid.

16. A method for evaluating efficacy of a cancer or tumor therapeutic agent comprising:
treating target drugs to the spheroid of claim 6; and
analyzing distributions of the target drugs in a core part of the spheroid or analyzing cell viability in the spheroid.

17. A method for evaluating efficacy of a cancer or tumor therapeutic agent comprising:
treating target drugs to the spheroid of claim 7; and
analyzing distributions of the target drugs in a core part of the spheroid or analyzing cell viability in the spheroid.

18. A method for evaluating efficacy of a cancer or tumor therapeutic agent comprising:
treating target drugs to the spheroid of claim 8; and
analyzing distributions of the target drugs in a core part of the spheroid or analyzing cell viability in the spheroid.
